(19) United States
(12) Patent Application Publication
(10) Pub. No.: US 2025/0331524 A1
(43) Pub. Date: Oct. 30, 2025

(54) YEAST STRAIN FOR FAST-FERMENTING SUGAR-FREE DOUGH AND USE THEREOF

(71) Applicant: ANGEL YEAST CO., LTD, Yichang, Hubei (CN)

(72) Inventors: Yafang SUN, Yichang, Hubei (CN); Minghua XIAO, Yichang, Hubei (CN); Liting CHEN, Yichang, Hubei (CN); Tianfen GUO, Yichang, Hubei (CN); Long WANG, Yichang, Hubei (CN); Jinbao KUANG, Yichang, Hubei (CN)

(21) Appl. No.: 18/870,073

(22) PCT Filed: Mar. 3, 2023
(86) PCT No.: PCT/CN2023/079577

§ 371 Date: Nov. 27, 2024

(30) Foreign Application Priority Data
May 27, 2022 (CN) 202210592367.1

(51) Int. Cl. A21D 8/04; A21D 13/062; C12N 1/18; C12R 1/865

(52) U.S. Cl. CPC A21D 8/047; A21D 13/062; C12N 1/185; C12R 2001/865

(57) ABSTRACT

The present invention provides a yeast strain for fast-fermenting sugar-free dough and uses thereof. The Saccharomyces cerevisiae AMCC31248 strain (Saccharomyces cerevisiae AMCC31248) provided by the present invention is deposited at the China Center for Type Culture Collection (CCTCC) with the deposit number CCTCC NO: M 20211686. The strain of Saccharomyces cerevisiae AMCC31248 strain provided by the present invention has good fermentation performance in sugar-free dough and can ferment sugar-free dough rapidly.

### TECHNICAL FIELD

The present invention relates to the field of microorganisms and in particular to a yeast strain for fast-fermenting sugar-free dough and use thereof.

### BACKGROUND ART

From the discovery of commercial baker's yeast to the formal formation of the yeast production process, to the development stage of active dry yeast nowadays, the variety of yeast products are increasingly diversified, and product quality are increasingly improved, and the yeast products can be used in the production of different flour food. As a kind of traditional staple food, fermented wheaten food occupies an important position in the people's dietary structure. According to statistics, about half of the population in China now takes fermented wheaten food as its staple food, and most of them are steamed bread, steamed stuffed buns and other fermented wheaten food fermented from dough without sugar. Sugar-free or low-sugared flour food is preferred by consumers from a health standpoint.

In flour food making, the dough fermentation process always plays a crucial role, not only affecting the softness, taste, nutritional value of the flour product, but also affecting the speed of the dough leavening. The stronger the yeast's fermentation ability, the faster the dough will rise, thereby shortening the fermentation cycle, speeding up the flour food-making process, and improving industrial production efficiency. Therefore, it is of great practical significance to develop Saccharomyces cerevisiae strains with good fermentation performance in sugar-free dough.

### SUMMARY OF THE INVENTION

During the flour food making process, the temperature of the environment is often too high, which causes the yeast to start too fast during kneading dough and causes the quality of the product to decline. In order to reduce the temperature of the dough, low-temperature water is usually added to reduce the temperature, but the yeast cells will enter into a shock state when they encounter a low-temperature environment, thus affecting the normal growth of yeast cells. There is therefore an urgent need for cold osmotic shock resistance of yeast.

Therefore, in view of the problems of low efficiency of fermenting sugar-free dough with Saccharomyces cerevisiae and poor cold osmotic shock resistance in the prior art, the present invention provides a yeast strain for fast-fermenting sugar-free dough with cold osmotic shock resistance.

In a first aspect, the present invention provides a Saccharomyces cerevisiae strain comprising:
Saccharomyces cerevisiae AMCC31248 strain (Saccharomyces cerevisiae AMCC31248) deposited at the China Center for Type Culture Collection (CCTCC) with the deposit number CCTCC NO: M 20211686.

In a second aspect, the present invention provides a fermentative preparation method for a Saccharomyces cerevisiae microbial agent, the process comprising the following steps: culturing the Saccharomyces cerevisiae strain.

Preferably, the preparation method comprises the following steps:
(1) amplifying and culturing the Saccharomyces cerevisiae strain;
(2) adding the product obtained in step (1) to a liquid culture medium, and fermenting and culturing at 26-32° C.

In a third aspect, the present invention provides a microbial agent containing the strain of Saccharomyces cerevisiae AMCC31248 strain (Saccharomyces cerevisiae AMCC31248).

Preferably, the microbial agent is obtained by the fermentative preparation method.

In a fourth aspect, the present invention also provides the use of the Saccharomyces cerevisiae strain or the microbial agent in fermentation.

In a fifth aspect, the present invention also provides the use of the microbial agent of the Saccharomyces cerevisiae strain in a dough.

In a sixth aspect, the present invention provides a dough containing the Saccharomyces cerevisiae strain or the microbial agent.

Preferably, the dough contains flour and the Saccharomyces cerevisiae strain in a mass ratio of 100:0.5-5.

In a seventh aspect, the present invention also provides a preparation method for a dough comprising the following steps: kneading the dough with water at 0-35° C.

The water at 0° C. in the present invention may be ice at 0° C. or a mixture of ice and water at 0°° C. or liquid water at 0° C.

In the preparation of the dough according to the present invention, the water, the flour, and the Saccharomyces cerevisiae may be added in any order, such as mixing the flour and the Saccharomyces cerevisiae and then adding water at 0-35° C. for kneading dough, adding the flour to water at 0-35° C. and then adding the Saccharomyces cerevisiae, or adding the Saccharomyces cerevisiae to water at 0-35° C. and then adding the flour.

In an eighth aspect, the present invention also provides a flour product obtainable by the dough preparation method.

Preferably, the flour product is a steamed bread, a steamed stuffed bun, a bread, a biscuit, a noodle, a pan-fried dumpling, and the like.

The present invention provides Saccharomyces cerevisiae AMCC31248 strain (Saccharomyces cerevisiae AMCC31248) provided by the present invention has good fermentation performance in sugar-free dough, can ferment sugar-free dough rapidly, and has good cold osmotic shock resistance.

### Strain Preservation Information

The Saccharomyces cerevisiae AMCC31248 strain (Saccharomyces cerevisiae AMCC31248) provided by the present invention was deposited at the China Center for Type Culture Collection (CCTCC) on Dec. 29, 2021, under the deposit number CCTCC NO: M 20211686, deposited at Wuhan University, Wuhan, China, postal code: 430072; TEL: 027-68754052.

The Saccharomyces cerevisiae AMCC30010 strain (Saccharomyces cerevisiae AMCC30010) used in the present invention was deposited at the China Center for Type Culture Collection (CCTCC) on Mar. 29, 2022, under the deposit number CCTCC NO: M 2022340, deposited at Wuhan University, Wuhan, China, postal code: 430072; TEL: 027-68754052.

The Saccharomyces cerevisiae AMCC32101 strain (Saccharomyces cerevisiae AMCC32101) provided by the present invention was deposited at the China Center for Type Culture Collection (CCTCC) on Mar. 29, 2022, with the deposit number CCTCC NO: M 2022341, deposited at Wuhan University, Wuhan, China, postal code: 430072; TEL: 027-68754052.

### BRIEF DESCRIPTION OF THE DRAWINGS

FIG. 1 shows a colony diagram of the strain Saccharomyces cerevisiae AMCC31248;

FIG. 2 shows a sporulation map of Saccharomyces cerevisiae strain AMCC31248;

FIG. 3 shows the growth curves of parental and new strains in a wort extract medium.

### DETAILED DESCRIPTION OF THE INVENTION

According to the present invention, a Saccharomyces cerevisiae AMCC31248 strain provided by the present invention is obtained by a micro hybridization method using a Saccharomyces cerevisiae AMCC30010 strain and a Saccharomyces cerevisiae AMCC32101 strain as parents.

Saccharomyces cerevisiae AMCC30010 (Saccharomyces cerevisiae

AMCC30010) strain is a Saccharomyces cerevisiae strain bred by Angel Yeast Co. Ltd. which was collected from Yichang City, Hubei Province. The cell morphology of the Saccharomyces cerevisiae strain was observed by light microscopy to be oval, budding, and a single colony growing on a solid plate with a slightly raised central spherical shape, milky white, smooth surface, and neat edge. Morphological observation and molecular biological identification by high-power microscope confirmed that it is a strain of Saccharomyces cerevisiae, which is a food product attribute. It was deposited with the China Center for Type Culture Collection (CCTCC) on Mar. 29, 2022, with the deposit number CCTCC NO: M 2022340 (i.e. CCTCC M 2022340).

Saccharomyces cerevisiae AMCC32101 (Saccharomyces cerevisiae AMCC32101) strain is a Saccharomyces cerevisiae strain bred by Angel Yeast Co. Ltd. which was collected from the city of Ulanqab, Inner Mongolia Autonomous Region. The cell morphology of the Saccharomyces cerevisiae strain was observed by light microscopy to be oval, budding, and a single colony growing on a solid plate with a slightly raised central spherical shape, milky white, smooth surface, and neat edge. Morphological observation and molecular biological identification by high-power microscope confirmed that it is a strain of Saccharomyces cerevisiae, which is a food product attribute. It was deposited with the China Center for Type Culture Collection (CCTCC) on Mar. 29, 2022, with the deposit number CCTCC NO: M 2022341 (i.e. CCTCC M 2022341).

The strain of Saccharomyces cerevisiae AMCC31248 strain provided by the present invention has good fermentation performance in sugar-free dough and can ferment sugar-free dough rapidly.

Some sugar-free bread, soda biscuits, and steamed bread are mainly made by sugar-free dough fermentation. Most of the flour is starch, which is converted into maltose under the action of amylase in flour. Therefore, the maltose utilization ability of yeast determines the rising speed of sugar-free dough. The maltose utilization enzymes of yeast include maltase and maltose permease. The yeast with high maltose utilization ability is called fast-fermenting yeast.

Sugar-tolerant yeast means that it has a higher tolerance to sucrose in sugar-containing doughs, i.e. the growth and fermentation properties in sugar-containing breads are higher than in normal yeasts.

Low sugar tolerant yeasts are used in about 7% sucrose dough systems, and high sugar tolerant yeasts are used in higher sucrose concentration dough systems, up to 25%. Sucrose is generally not directly available to microorganisms, whereas Saccharomyces cerevisiae contains a sucrose hydrolyzing enzyme that degrades sucrose, which acts on the beta-1,2 glycosidic linkages to hydrolyze sucrose to D-glucose and D-fructose, which then enters the glycolytic pathway for use by the yeast, while the glucose and fructose produced by the rapid decomposition of sucrose increase the osmotic pressure around the yeast cells. The yeast cell membrane is a selective semi-permeable biofilm. The activity of yeast cells is affected by the concentration of the external environment. When the cells are in a high osmotic pressure environment, the water content and protoplast in the cells will leak out of the cell membrane to make the cells dehydrated and even die. Therefore, the high osmotic environment that Saccharomyces cerevisiae faces in high-sugar dough has an effect on its growth and fermentation performance. Therefore, the gas production capacity of sugar-free yeast is determined by maltose utilization enzyme activity, and the gas production capacity of sugar-tolerant yeast is determined by sucrase activity.

The reagents and instrument source information used in the examples of the present invention are shown in the following Tables 1 and 2.

TABLE 1
Reagents information table

| Reagents | Vendor |
|---|---|
| Yeast extract powder | Angel yeast |
| Peptone | Angel yeast |
| Glucose | SCR, SINOPHARM |
| Agar | Huixing |
| Potassium acetate | SCR, SINOPHARM |

TABLE 2
Instrument information sheet

| Instrument | Model | Vendor |
|---|---|---|
| Constant temperature | ZWYR-2102C | Shanghai Zhicheng |
| shaker | | |
| Biochemical incubator | SPX-150BIII | Tianjin Taiste |
| Bechtop | SKJH-1109 | Shanghai Sukun |
| Clean bench | ME4002E | METTLER TOLEDO |
| pH meter | PB-10 | Sartorius |
| Thermostat water bath | HH-2 | Jiangsu Guohua |
| Centrifuge | DL-5200B | Shanghai Anting |
| Rapid moisture meter | MJ33 | METTLER TOLEDO |
| PCR instrument | C1000 | BIO-RAD |
| Gel imaging system | Gel Doc ™ XR⁺ | BIO-RAD |
| Electrophoresis apparatus | EPS-300 | Shanghai Tianneng |
| Light microscope | CX43 | OLYMPUS |
| Fully automatic growth | BioscreenC | OY Growth Curves |
| curve analyzer | | |
| Yeast micromanipulator | MSM 400 | SINGER |

The formulation of the sporulation medium used in the examples of the present invention was: 1% potassium acetate, 0.1% yeast extract powder, 0.05% glucose, and 2% agar.

In the examples of the present invention, each Saccharomyces cerevisiae strain was activated using a YPD solid medium, the formulation of the YPD solid medium was as follows: 1% yeast extract powder, 2% peptone, 2% glucose, and 2% agar.

In the examples of the present invention, each Saccharomyces cerevisiae strain was cultured in a YPD liquid medium: the formulation of the YPD liquid medium was as follows: 1% yeast extract powder, 2% peptone, and 2% glucose.

### Example 1 Strain Construction and Identification

The parent strains Saccharomyces cerevisiae AMCC30010 strain and Saccharomyces cerevisiae AMCC32101 strain were activated and induced to sporulate, respectively. After the enzymolysis of bacterial cells, the single spores were picked using a yeast micromanipulator, and the single spores from two different parents were contacted, and then cultured at 30° C. The spore morphology was observed, and the strain was continued to incubate at 30° C. after two single spores were successfully hybridized. This was a first-generation strain. The first-generation strains were further crossed with the Saccharomyces cerevisiae AMCC30010 strain to obtain the second-generation strains, which were then tested for sporulation, and heterozygous strains were selected for subsequent screening.

The growth curve of the new heterozygous strains was determined by the fully automatic growth curve analyzer Bioscreen C, and the heterozygous strains with higher growth efficiency than their parents and ranked first 20 were selected.

A shake flask fermentation test was performed on 20 heterozygous strains with higher growth efficiency than their parents. The net dry weight of the strain and the fermentation activity of fresh yeast in the 0% sugar dough system were used as screening indicators, heterozygous strains with net dry weight of yeast milk reaching 95-105% of either parent and 0% sugar dough fermentation activity reaching 95%-150% of either parent were selected.

Subsequently, the heterozygous strains obtained by the above-mentioned screening step were cultured in a 45 L fermenter system, and the obtained yeast cells were respectively prepared into active dry yeasts, and the fermentation activity of the active dry yeasts in a 0% sugar dough system was determined, and a new strain was selected which has no obvious abnormality during the preparation of the dry yeasts, and the 0% sugar dough fermentation activity of the active dry yeasts can reach 95%-120% of that of any parent.

Finally, the heterozygous strains preferred in the above-mentioned steps were screened for cold osmotic shock resistance. 0% sugar dough containing heterozygous strain active dry yeast was prepared with crushed ice and flour at 0° C. by kneading dough, and the leavening time of the dough was determined. The heterozygous strain with the shortest leavening time of the corresponding dough was used as the target strain to screen out the heterozygous strain with cold osmotic shock resistance.

A heterozygous strain named AMCC31248 was obtained by the above-mentioned screening, which had high fermentation activity in 0% sugar dough and good cold osmotic shock resistance. The strain was identified and the identification result was:

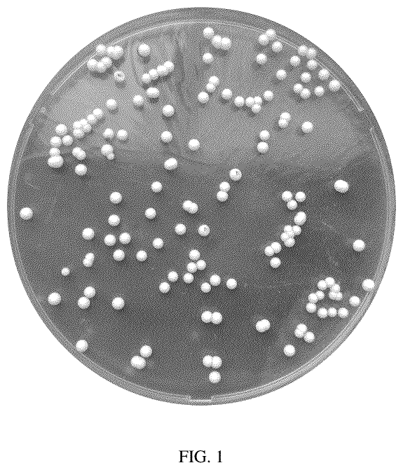

Under the light microscope, it was observed that the cell morphology of the strain was oval, sprouting, and growing. The single colony grown on the solid plate was in a slightly raised central spherical shape, milky white, loose texture, easily lifted by the inoculation loop, smooth surface, drier and neat edge. FIG. 1 shows the colony diagram of heterozygous strain AMCC31248.

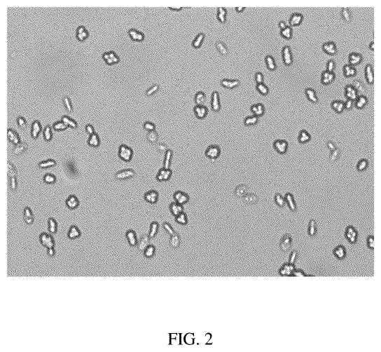

Microscopic examination of sporulation of the heterozygous strain AMCC31248 was shown in FIG. 2, which showed that the heterozygous strain had sporulation ability as indicated by more spores and full morphology in the microscopic field.

The resulting heterozygous strain AMCC31248 was named Saccharomyces cerevisiae AMCC31248 strain (Saccharomyces cerevisiae AMCC31248). This Saccharomyces cerevisiae strain AMCC31248 was deposited at the China Center for Type Culture Collection (CCTCC) on Dec. 29, 2021, with the deposit number CCTCC NO: M 20211686 (i.e. CCTCC M 20211686).

### Example 2 Growth Efficiency Determination

The Saccharomyces cerevisiae AMCC31248 strain obtained in Example 1 and parent strains Saccharomyces cerevisiae AMCC30010 and Saccharomyces cerevisiae AMCC32101 were inoculated into wort extract medium (purchased from Hope

Bio-Technology Co., Ltd), cultured at 30° C. for 48 h, and the OD₆₀₀ value of each strain at different times were determined by high-throughput analysis using the fully automatic growth curve analyzer Bioscreen C. The growth curve was drawn with time (h) as abscissa and the corresponding OD₆₀₀ value as ordinate. The test data were analyzed and the growth efficiency of the strains was calculated according to the following formula.
Growth efficiency = (OD2-OD1)/(t2-t1)
OD1: OD₆₀₀ value corresponding to strain at t1;
OD2: OD₆₀₀ value corresponding to strain at t2;
t1: the start of the logarithmic growth phase;
t2: the end of the logarithmic growth phase.

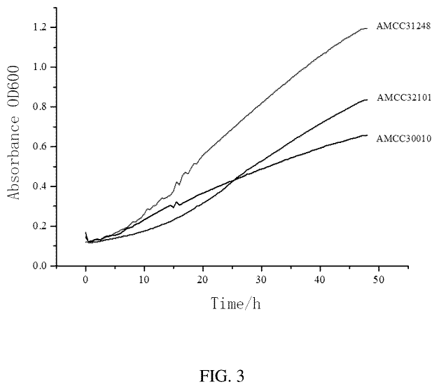

The growth curve of Saccharomyces cerevisiae AMCC31248 is shown in FIG. 3. It can be seen that the strain grows rapidly in the wort extract medium. The growth efficiency of the parent strain and the Saccharomyces cerevisiae AMCC31248 strain is shown in Table 3 below.

TABLE 3
Growth efficiency data of parent and new strains

| Strain name | Growth efficiency |
|---|---|
| AMCC30010 | 0.013 |
| AMCC32101 | 0.017 |
| AMCC31248 | 0.028 |

As can be seen from above Table 3, the growth efficiency of the obtained Saccharomyces cerevisiae AMCC31248 strain was significantly higher than that of the parent strains Saccharomyces cerevisiae AMCC30010 and Saccharomyces cerevisiae AMCC32101.

### Example 3 Fresh Yeast Fermentation Activity Assay

The Saccharomyces cerevisiae AMCC31248 obtained in Example 1 was inoculated into a shake flask containing a fermentation medium and cultured at 30° C. The precipitate collected after centrifugation, i.e. the yeast milk, was weighed and the water content of the yeast milk was determined. The net dry weight (g/L) of each strain in the shake flask was calculated according to the following formula: Net dry weight (g/L)=weight of yeast milk×(1−water content)

According to the 0% sugar dough system shown in Table 4, the dough was prepared to test the fermentation activity of the Saccharomyces cerevisiae AMCC31248 strain, the mass of yeast milk of Saccharomyces cerevisiae AMCC31248 strain and parent strains Saccharomyces cerevisiae AMCC30010 and Saccharomyces cerevisiae AMCC32101 required to be added was calculated and weighed, respectively; according to the dough formulation shown, flour, salt, and water were weighed respectively, and mixed in a dough kneader to prepare the dough; an SJA fermenter was used to directly determine the total volume of carbon dioxide gas produced by yeast fermentation of 280 g dough prepared by the system shown in Table 4 at 30° C. for 1 h, i.e. the fermentation activity of the strain, and the results were expressed in milliliters (mL).

TABLE 4
0% Sugar Dough System

| Raw material | Flour/g | Salt/g | Water/g | Dry yeast/g |
|---|---|---|---|---|
| Addition | 280 | 4 | 144 | 2.8 |
| amount | | | | |

The relative percentage of net dry weight in Table 5 was calculated according to the following formula:
Relative percentage of net dry weight (%)=(Net dry weight of heterozygous new strain/Net dry weight of parental strain)*100%

The relative percentage of dough fermentation activity of the Saccharomyces cerevisiae AMCC31248 strain compared to the parent strain in Table 6 was calculated according to the following formula:
Relative percentage of dough fermentation activity (%)=dough fermentation activity of hybrid new strain/dough fermentation activity of parent strain*100%

As shown in Table 5, the net dry weight of the Saccharomyces cerevisiae AMCC31248 strain was 95.7% and 109.9% of that of parent strains Saccharomyces cerevisiae AMCC30010 and Saccharomyces cerevisiae AMCC32101, respectively. As shown in Table 6, the 0% sugar dough fermentation activity of the Saccharomyces cerevisiae AMCC31248 strain was superior to both parent strains, with an advantage of about 10%.

TABLE 5
Net dry weight data of parent and new strains

| | Net dry | Relative | Relative |
|---|---|---|---|
| Strain name | weight/(g/L) | percentage % | percentage % |
| AMCC30010 | 13.37 | 100.0 | 114.8 |
| AMCC32101 | 11.65 | 87.1 | 100.0 |
| AMCC31248 | 12.80 | 95.7 | 109.9 |

TABLE 6
Fermentation activity data of fresh yeast in 0% sugar dough system

| | 0% sugar 1 h | Relative | Relative |
|---|---|---|---|
| Strain name | activity/mL | percentage % | percentage % |
| AMCC30010 | 1050 | 100.0 | 103.1 |
| AMCC32101 | 1018 | 97.0 | 100.0 |
| AMCC31248 | 1145 | 109.0 | 112.5 |

### Example 4 Active Dry Yeast Fermentation Activity Assay

The strain AMCC31248 of Saccharomyces cerevisiae was activated and cultured in a 45 L fermenter. Then the activated dry yeast was isolated, washed, filtered, and dried. Flour, salt, water, and prepared active dry yeast were respectively weighed according to the dough formulation shown in Table 7, and the total amount of carbon dioxide gas produced by fermenting 280 g of the dough prepared by the system shown in Table 7 for 1 h by the active dry yeast was assayed using the SJA method, i.e. the dough fermentation activity of the active dry yeast.

TABLE 7
0% Sugar dough system

| Raw material | Flour/g | Salt/g | Water/g | Dry yeast/g |
|---|---|---|---|---|
| Addition | 280 | 4 | 144 | 2.8 |
| amount | | | | |

Since it is difficult to dry the parent strain AMCC32101 during the preparation of active dry yeast and there is no corresponding fermentation activity data, the dry yeast activity of parent strain AMCC30010 is only used as the control here. There was no obvious abnormality in the preparation of dry yeast of Saccharomyces cerevisiae AMCC31248 strain, and the specific data of 0% sugar dough fermentation activity assay of active dry yeast were shown in Table 8. Among them, the calculation method of the relative percentage of fermentation activity in Table 8 is as follows:

The relative percentage of fermentation activity=(fermentation activity of hybrid new strain/fermentation activity of parent strain AMCC 30010)*100%

TABLE 8
Fermentation activity data of active
dry yeast in 0% sugar dough system

| | 0% sugar 1 h | Relative percentage of |
|---|---|---|
| Strain name | activity/mL | fermentation activity/% |
| AMCC30010 | 1016 | 100.0 |
| AMCC31248 | 1133 | 111.5 |

The results showed that compared with the parent Saccharomyces cerevisiae AMCC30010 strain, the Saccharomyces cerevisiae AMCC31248 strain still had an 11.5% advantage in dough fermentation activity under the condition of 0% sugar, indicating that Saccharomyces cerevisiae AMCC31248 strain had certain resistance to drying.

### Example 5 Cold Osmotic Shock Resistance Assay of Active Dry Yeast

According to the dough formulation shown in Table 9, 0° C. crushed ice was poured into the dough kneader, active dry yeast was added to mix at low speed, then flour and salt were added, continued mixing and stirring until uniform, and the dough was prepared. After shaping 400 g of dough, it was placed in a leavening box for fermentation, the temperature was controlled to 38+1° C., the humidity was controlled to 85-90%, and the time required for dough fermentation to reach the same set height was recorded, namely, the fermentation time.

TABLE 9
Cold osmotic shock dough systems

| Raw material | Flour/g | Salt/g | Dry yeast/g | Crushed ice/g |
|---|---|---|---|---|
| Addition | 100 | 2 | 1 | 63 |
| amount | | | | |

The fermentation time of the dough corresponding to the strain of Saccharomyces cerevisiae AMCC31248 and the parent strain of Saccharomyces cerevisiae AMCC30010 is shown in Table 10. Among them, the calculation method of the relative percentage of fermentation time in Table 10 is as follows:

The relative percentage of fermentation time=(fermentation time of hybrid new strain/fermentation time of parent strain AMCC30010)*100%

TABLE 10
Fermentation time data of active dry yeast
in cold osmotic shock dough system

| | Fermentation | Relative percentage of |
|---|---|---|
| Strain name | time/min | fermentation time/% |
| AMCC30010 | 154.3 | 100.0 |
| AMCC31248 | 108.0 | 70.0 |

The results showed that the fermentation time of the Saccharomyces cerevisiae AMCC31248 strain was only 70.0% of that of the parent Saccharomyces cerevisiae AMCC30010 strain, with an advantage of 30%, indicating that Saccharomyces cerevisiae AMCC31248 strain grew faster and had excellent cold osmotic shock resistance under the same conditions.

While the present invention has been described in connection with what is presently considered to be the most practical and preferred examples, it is to be understood that the present invention is not limited to the disclosed embodiments, but, on the contrary, is intended to cover various modifications and equivalent arrangements included within the spirit and scope of the appended claims.

1. A Saccharomyces cerevisiae strain, characterized in that the Saccharomyces cerevisiae strain is:
Saccharomyces cerevisiae AMCC31248 strain (Saccharomyces cerevisiae AMCC31248) deposited at the China Center for Type Culture Collection (CCTCC) with the deposit number CCTCC NO: M 20211686.

2-3. (canceled)

4. A microbial agent, comprising the Saccharomyces cerevisiae AMCC31248 strain (Saccharomyces cerevisiae AMCC31248) according to claim 1.

5. The microbial agent according to claim 4, characterized in that the microbial agent is obtained by culturing the Saccharomyces cerevisiae strain according to claim 1.

6.-7. (canceled)

8. A dough, comprising the Saccharomyces cerevisiae strain according to claim 1.

9. The dough according to claim 8, characterized in that the dough comprises flour and the Saccharomyces cerevisiae strain in a mass ratio of 100:0.5-5.

10.-12. (canceled)

13. The microbial agent according to claim 4, characterized in that the microbial agent is obtained by a method comprising the following steps:
(1) amplifying and culturing the Saccharomyces cerevisiae strain according to claims 1; and
(2) adding the product obtained in step (1) to a liquid medium, and fermenting and culturing at 26-32° C.

14. The dough according to claim 8, characterized in that the dough is used for the preparation of a steamed bread, a steamed stuffed bun, a bread, a biscuit, a noodle, or a pan-fried dumpling.

15. The dough according to claim 9, characterized in that the dough is used for the preparation of a steamed bread, a steamed stuffed bun, a bread, a biscuit, a noodle, or a pan-fried dumpling.

16. The dough according to claim 8, characterized in that the dough is a sugar-free dough.

17. The dough according to claim 9, characterized in that the dough is a sugar-free dough.

18. The dough according to claim 14, characterized in that the dough is a sugar-free dough.

19. The dough according to claim 15, characterized in that the dough is a sugar-free dough.
